(19) United States
(12) Patent Application Publication
(10) Pub. No.: US 2026/0124363 A1
(43) Pub. Date: May 7, 2026

(54) INFUSION SYSTEM AND PUMP WITH CONFIGURABLE CLOSED LOOP DELIVERY RATE CATCH-UP

(71) Applicant: ICU Medical, Inc., San Clemente, CA (US)

(72) Inventors: Steve Joseph Lindo, Chicago, IL (US); William Kenneth Day, Hoffman Estates, IL (US)

(21) Appl. No.: 19/381,417

(22) Filed: Nov. 6, 2025

Related U.S. Application Data
(63) Continuation of application No. 17/804,548, filed on May 27, 2022, now Pat. No. 12,485,221.
Application No. 17/804,548 is a continuation of application No. 14/725,077, filed on May 29, 2015, now Pat. No. 11,344,673.
(60) Provisional application No. 62/004,688, filed on May 29, 2014.

(51) Int. Cl. A61M 5/168; A61M 5/172

(52) U.S. Cl. CPC A61M 5/16827; A61M 5/1723

(57) ABSTRACT

An infusion system and pump with configurable closed loop delivery rate catch-up including an infusion system having a medication management unit and a medical device. The medication management unit has programming code to: provide a graphical user interface for modifying a drug library; receive a catch-up rate factor; update the drug library with the catch-up rate factor; and transmit the updated drug library to the medical device. The medical device has programming code to: receive the updated drug library; receive a desired infusion rate; calculate expected accumulated infusion volume from the desired infusion rate; request delivery of the infusion at the desired infusion rate; determine actual accumulated infusion volume at a particular time; increase the infusion rate by the catch-up rate factor to generate a catch-up infusion rate when at the particular time the actual accumulated infusion volume is less than expected; and request infusion at the catch-up infusion rate.

### TECHNICAL FIELD

The present invention relates to medical devices. More specifically, the invention relates to infusion systems and pumps with configurable closed loop delivery rate catch-up.

### BACKGROUND OF THE INVENTION

### Description of the Related Art

Infusion pumps are medical devices that deliver fluids, including nutrients and medications such as antibiotics, chemotherapy drugs, and pain relievers, into a patient's body in controlled amounts. Many types of pumps, including large volume, patient-controlled analgesia (PCA), elastomeric, syringe, enteral, and insulin pumps, are used worldwide in healthcare facilities such as hospitals, and in the home. Clinicians and patients rely on pumps for safe and accurate administration of fluids and medications.

Presently available infusion pumps use an open loop pumping rate: the desired pumping or volumetric flow rate is input directly, or calculated from an input volume to be infused and delivery period or duration, and the infusion pump operates at a single target motor speed or stroke frequency to deliver the desired pumping or flow rate regardless of external conditions. Unfortunately, flow delivery can be interrupted by a variety of conditions, such as a stoppage or pause based upon a full or partial occlusion, a kinked tube, an air-in-line alarm, hanging a new IV bag, vein clot, or the like. Once the flow delivery is interrupted, the time in which there is no medication delivery is lost, resulting in a delay in desired infusion completion.

Nurses typically work in shifts and expect certain medications to be started and/or finished within their shift and plan accordingly. When occlusions, pauses, or other disturbances interrupt or delay medication delivery, this disrupts the nurses'planning for patient care within their respective shifts. In addition, the patients in these scenarios would not receive the medicine required within the allotted time.

It would be desirable to have an infusion system and pump with configurable closed loop delivery rate catch-up that would overcome the above disadvantages.

### SUMMARY OF THE INVENTION

One aspect of the present invention provides an infusion system with catch-up of interrupted delivery of an infusion programmable by a user, the infusion system including a medication management unit having a processing unit and a storage medium coupled to the processing unit, the storage medium containing programming code executable by the processing unit to: provide a graphical user interface for modifying a drug library of the medication management unit; receive a catch-up rate factor on the graphical user interface; update the drug library with the catch-up rate factor; and transmit the updated drug library to a memory of an infusion pump; and the infusion pump being in electronic communication with the medication management unit, having a processor and the memory being coupled to the processor, the memory containing programming code executable by the processor to: receive the updated drug library at the medical device with the catch-up rate factor; receive a desired infusion rate from the user at the medical device; calculate an expected accumulated infusion volume as a function of time from the desired infusion rate; request delivery of the infusion at the desired infusion rate; determine an actual accumulated infusion volume at a particular time; increase the desired infusion rate by the catch-up rate factor to generate a catch-up infusion rate when at the particular time the actual accumulated infusion volume is less than the expected accumulated infusion volume; and request delivery of the infusion at the catch-up infusion rate.

Another aspect of the present invention provides an infusion pump with catch-up of interrupted delivery of an infusion programmable by a user, the infusion pump including a processor; a memory coupled to the processor, the memory containing programming code to: receive a desired infusion rate from the user at the medical device; calculate an expected accumulated infusion volume as a function of time from the desired infusion rate; request delivery of the infusion at the desired infusion rate; determine an actual accumulated infusion volume at a particular time; increase the desired infusion rate by a catch-up rate factor to generate a catch-up infusion rate when at the particular time the actual accumulated infusion volume is less than the expected accumulated infusion volume; and request delivery of the infusion at the catch-up infusion rate.

Yet another aspect of the present invention provides a method of catching-up interrupted delivery of an infusion from an infusion pump, the method including entering a desired infusion rate for the infusion pump; calculating an expected accumulated infusion volume as a function of time from the desired infusion rate; requesting the infusion pump to deliver the infusion at the desired infusion rate; determining an actual accumulated infusion volume at a particular time; increasing the (desired) infusion rate by a catch-up rate factor to generate a catch-up infusion rate when at the particular time the actual accumulated infusion volume is less than the expected accumulated infusion volume; and requesting the infusion pump to deliver the infusion at the catch-up infusion rate.

The foregoing and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is a schematic diagram of the medication management system including a medication management unit and a medical device integrated with other systems in a hospital environment, in accordance with the present invention;

FIG. 2 is a schematic diagram of the medication management unit, in accordance with the present invention;

FIG. 3 is a schematic diagram of some of the major functions performed by the medication management unit, in accordance with the present invention;

FIG. 4 is a schematic diagram of a medical device, in accordance with the present invention;

FIGS. 5A & 5B are perspective views of a multi-channel medical device, in accordance with the present invention;

FIGS. 6A & 6B are screen shots of a graphical user interface and detail of the graphical user interface, respectively, for configuring a drug library, in accordance with the present invention;

FIG. 7 is a graph of an infusion volume and infusion rate versus time modeled for an infusion with an infusion pump employing configurable closed loop delivery rate catch-up, in accordance with the present invention;

FIG. 8 is a block diagram of a control model for an infusion pump employing configurable closed loop delivery rate catch-up, in accordance with the present invention;

FIG. 9 is a flowchart of a method for configuring a drug library for use with an infusion system employing configurable closed loop delivery rate catch-up, in accordance with the present invention; and

FIG. 10 is a flowchart of a method for operating an infusion pump employing configurable closed loop delivery rate catch-up, in accordance with the present invention.

### DETAILED DESCRIPTION OF THE PREFERRED EMBODIMENT

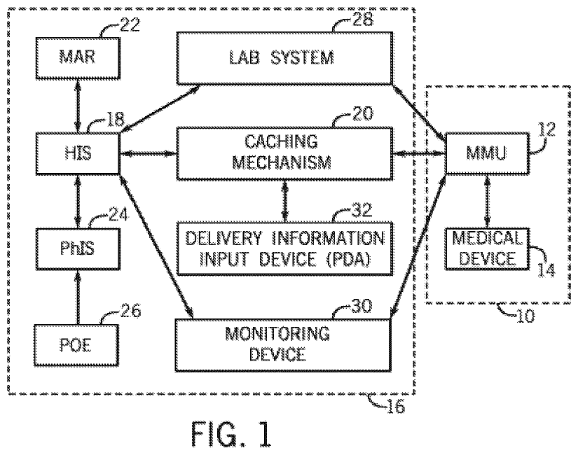

FIG. 1 is a schematic diagram of the medication management system including a medication management unit and a medical device integrated with an information system, in accordance with the present invention. The medication management system (MMS) 10 includes a medication management unit (MMU) 12 and a medical device 14, typically operating in a hospital environment 16. The term hospital environment as defined herein is used broadly to mean any medical care facility, including but not limited to a hospital, treatment center, clinic, doctor's office, day surgery center, hospice, nursing home, and any of the above associated with a home care environment. There can be a variety of information systems in a hospital environment. As shown in FIG. 1, the MMU 12 communicates to a hospital information system (HIS) 18 via a caching mechanism 20 that is part of the hospital environment 16.

Those skilled in the art will appreciate that the caching mechanism 20 is primarily a pass through device for facilitating communication with the HIS 18 and its functions can be eliminated or incorporated into the MMU 12 (FIG. 1) and/or the medical device 14 and/or the HIS 18 and/or other information systems or components within the hospital environment 16. The caching mechanism 20 provides temporary storage of hospital information data separate from the HIS 18, the medication administration record system (MAR) 22, pharmacy information system (PhIS) 24, physician order entry (POE) 26, and/or Lab System 28. The caching mechanism 20 provides information storage accessible to the medication management system 10 to support scenarios where direct access to data within the hospital environment 16 is not available or not desired. In one example, the caching mechanism 20 provides continued flow of information in and out of the MMU 12 in instances where the HIS 18 is down or the connectivity between the MMU 12 and the electronic network (not shown) is down.

The HIS 18 communicates with a medication administration record system (MAR) 22 for maintaining medication records and a pharmacy information system (PhIS) 24 for delivering drug orders to the HIS. A physician/provider order entry (POE) device 26 permits a healthcare provider to deliver a medication order prescribed for a patient to the hospital information system directly or indirectly via the PhIS 24. One skilled in the art will also appreciate that a medication order can be sent to the MMU 12 directly from the PhIS 24 or POE device 26. As used herein, the term medication order is defined as an order to administer something that has a physiological impact on a person or animal, including but not limited to liquid or gaseous fluids, drugs or medicines, liquid nutritional products and combinations thereof.

Lab system 28 and monitoring device 30 also communicate with the MMU 12 to deliver updated patient-specific information to the MMU 12. As shown, the MMU 12 communicates directly to the lab system 28 and monitoring device 30. However, those skilled in the art will appreciate that the MMU 12 can communicate with the lab system 28 and monitoring device 30 indirectly via the HIS 18, the caching mechanism 20, the medical device 14 or some other intermediary device or system.

Delivery information input device 32 also communicates with the MMU 12 to assist in processing drug orders for delivery through the MMU 12. The delivery information input device 32 can be any sort of data input means, including those adapted to read machine readable indicia such as barcode labels; for example a personal digital assistant (PDA) with a barcode scanner. Hereinafter, the delivery information input device 32 is referred to as input device 32. Alternatively, the machine readable indicia can be in other known forms, such as radio frequency identification (RFID) tag, two-dimensional bar code, ID matrix, transmitted radio ID code, human biometric data such as fingerprints, etc. and the input device 32 adapted to “read” or recognize such indicia. The input device 32 is shown as a separate device from the medical device 14; alternatively, the input device 32 communicates directly with the medical device 14 or can be integrated wholly or in part with the medical device.

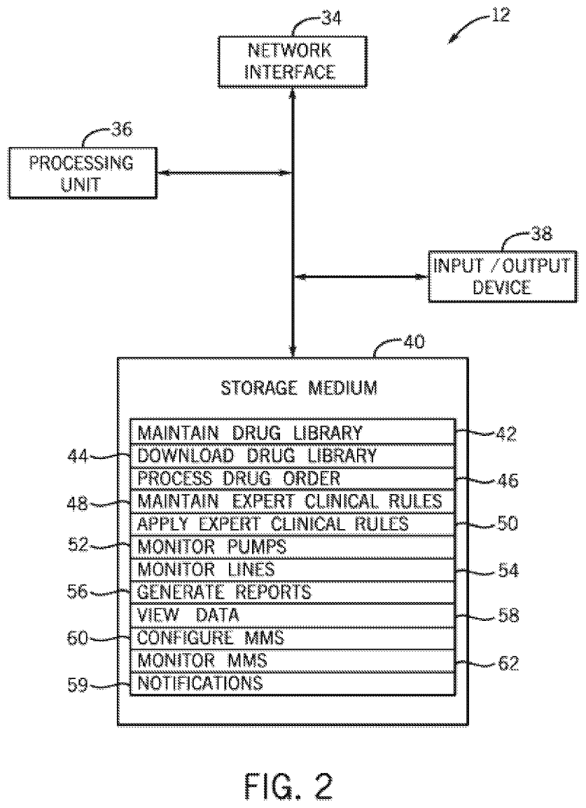

FIG. 2 is a schematic diagram of the medication management unit, in accordance with the present invention. The medication management unit 12 includes a network interface 34 for connecting the MMU 12 to multiple components of a hospital environment 16, one or more medical devices 14, and any other desired device or network. A processing unit 36 is included in MMU 12 and performs various operations described in greater detail below. A display/input or user interface device 38 communicates with the processing unit 36 and allows the user to receive output from processing unit 36 and/or input information into the processing unit 36. Those skilled in the art will appreciate that the display/input device 38 can be provided as a separate display device and a separate input device.

An electronic storage medium 40 communicates with the processing unit 36 and stores programming code and data necessary for the processing unit 36 to perform the functions of the MMU 12. More specifically, the storage medium 40 stores multiple programs formed in accordance with the present invention for various functions of the MMU 12 including but not limited to the following programs: Maintain Drug Library 42; Download Drug Library 44; Process Drug Order 46; Maintain Expert Clinical Rules 48; Apply Expert Clinical Rules 50; Monitor Pumps 52; Monitor Lines 54; Generate Reports 56; View Data 58; Configure the MMS 60; and Monitor the MMS 62. The Maintain Drug Library 42 program creates, updates, and deletes drug entries and establishes a current active drug library. The Download Drug Library 44 program updates medical devices 14 with the current drug library. The Process Drug Order 46 program processes the medication order for a patient, verifying that the point of care (POC) medication and delivery parameters match those ordered. The Maintain Expert Clinical Rules 48 program creates, updates, and deletes the rules that describe the hospital's therapy and protocol regimens. The Apply Expert Clinical Rules 50 program performs logic processing to ensure safety and considers other infusions or medication orders, patient demographics, and current patient conditions. The Monitor Pumps 52 program acquires ongoing updates of status events, and alarms transmitted both near real-time and in batch mode, as well as tracking the location, current assignment, and software versions such as the drug library version residing on medical device 14. The Monitor Lines 54 program acquires ongoing updates of status, events and alarms for each channel or line for a medical device 14 that supports multiple drug delivery channels or lines. The Generate Reports 56 program provides a mechanism that allows the user to generate various reports of the data held in the MMU storage medium 40. The View Data 58 program provides a mechanism that supports various display or view capabilities for users of the MMU 12. The Notifications 59 program provides a mechanism for scheduling and delivery of events to external systems and users. The Configure the MMS 60 program provides a mechanism for system administrators to install and configure the MMS 10. The Monitor the MMS 62 program enables information technology operations staff capabilities to see the current status of MMS 10 components and processing, and other aspects of day-to-day operations such as system start up, shut down, backup and restore.

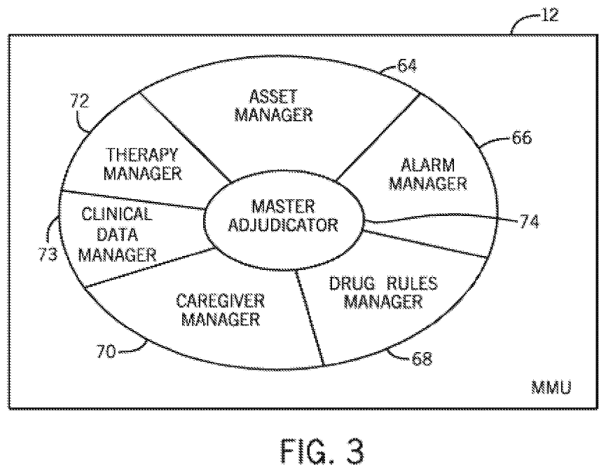

FIG. 3 is a schematic diagram of some of the major functions performed by the medication management unit, in accordance with the present invention. The various functional programs 42-62 of the MMU 12, each including separate features and rules, are partitioned (at a higher level than shown in FIG. 2) and logically organized into interrelated managing units of the MMU 12. As shown, the MMU 12 includes an asset manager 64, an alarm manager 66, a drug library manager (such as, for example, is included in HOSPIRA MEDNET™ software) 68, a caregiver manager 70, a therapy manager 72, and/or a clinical data manager 73. However, those skilled in the art will appreciate that additional or alternative hospital system managing units can be provided without departing from the present invention. Additionally, the MMU 12 includes a master adjudicator 74 between the separate interrelated managing units 64-73 of the MMU 12, to regulate the interaction between the separate management units.

Further, while the MMU 12 as described herein appears as a single device, there can be more than one MMU 12 operating harmoniously and sharing the same database. For example the MMU 12 can consist of a collection of MMU specific applications running on distinct servers in order to avoid a single point of failure, address availability requirements, and handle a high volume of requests. In this example, each individual server portion of the MMU 12 operates in conjunction with other server portions of the MMU 12 to redirect service requests to another server portion of the MMU 12. Additionally, the master adjudicator 74 assigns redirected service requests to another server portion of the MMU 12, prioritizing each request and also ensuring that each request is processed.

With reference to FIGS. 2 & 3, the managing units 64-72 each include separate features and rules to govern their operation. For example, the asset manager 64 governs the execution of the Monitor Pumps 52 and Monitor Lines 54 programs; the drug library manager 68 governs the execution of the Drug Library 42 and Download Drug Library 44 programs; the therapy manager 72 governs the execution of the Process Drug Order 46, Maintain Expert Clinical Rules 48, and Apply Expert Clinical Rules 50 programs; and the clinical data manager 73 governs the execution of the Generate Reports 56 and View Data 58 programs. Other distribution of the functional MMU programs 42-62 among the managing units 64-73 can be made in accordance with the present invention.

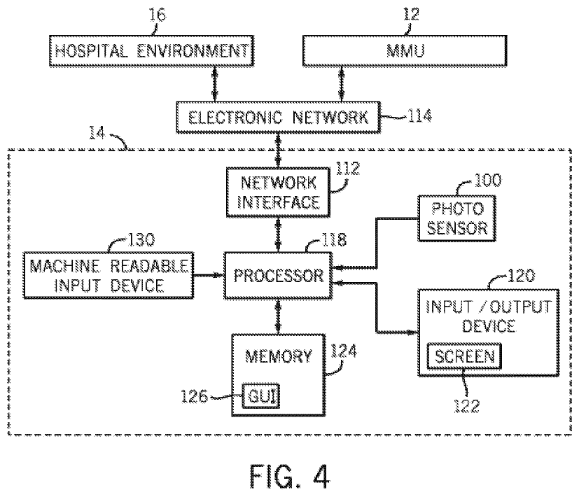

FIG. 4 is a schematic diagram of a medical device, in accordance with the present invention. An electronic network 114 connects the MMU 12, medical device 14, and hospital environment 16 for electronic communication. The electronic network 114 can be a completely wireless network, a completely hard wired network, or some combination thereof. As used herein, the term “medical device” includes without limitation a device that acts upon a cassette, reservoir, vial, syringe, or tubing to convey medication or fluid to or from a patient (for example, an enteral pump, a parenteral infusion pump, a patient controlled analgesia (PCA) or pain management medication pump, or a suction pump), a monitor for monitoring patient vital signs or other parameters, or a diagnostic, testing or sampling device.

The pump style medical device 14 includes a network interface 112 for connecting the medical device 14 to electronic network 114. Where a wireless connection to the electronic network 114 is desired, network interface 112 operates an antenna for wireless connection to the electronic network 114. The antenna can project outside the medical device 14 or be enclosed within the housing of the device.

A processor 118 is included in the medical device 14, includes a real time clock (not shown), and performs various operations described in greater detail below. The input/output device 120 allows the user to receive output from the medical device 14 and/or input information into the medical device 14.

Those skilled in the art will appreciate that input/output device 120 can be provided as a single device such as a touch screen 122, or as a separate display device and a separate input device (not shown). In one embodiment, the display screen 122 of the medical device 14 is a thin film transistor active matrix color liquid crystal display with a multi-wire touch screen. A membrane generally impermeable to fluids overlays the display screen 122 so the user can press images of keys or buttons on the underlying screen with wet gloves, dry gloves, or without gloves to trigger an input.

A memory 124 communicates with the processor 118 and stores code and data necessary for the processor 118 to perform the functions of the medical device 14. More specifically, the memory 124 stores multiple programs formed in accordance with the present invention for various functions of the medical device 14 including a graphical user interface program 126 with multiple subparts described in greater detail below.

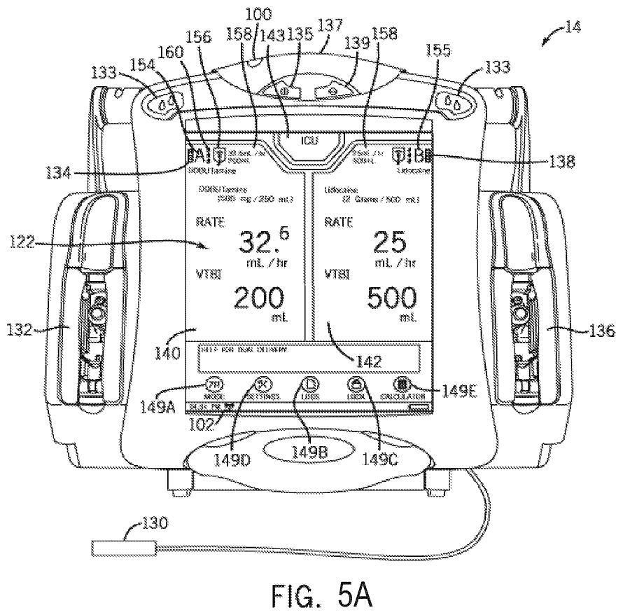

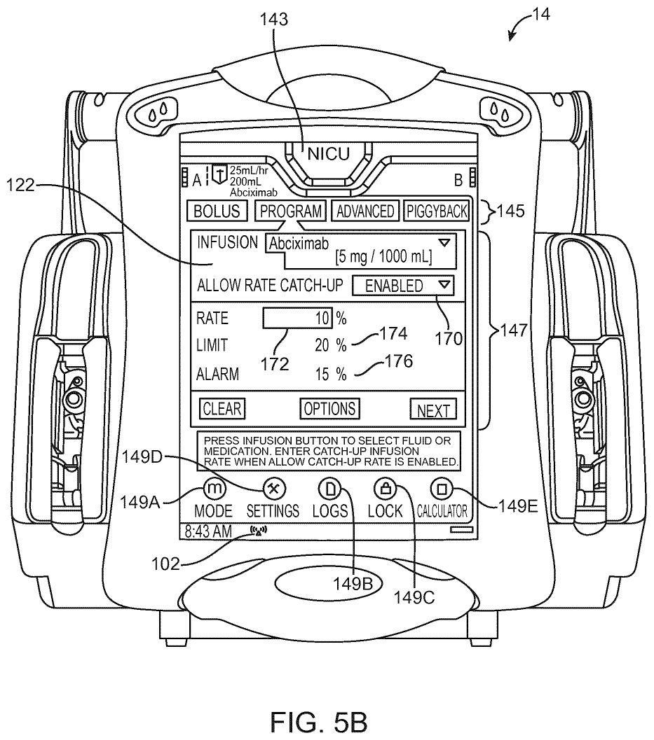

FIGS. 5A & 5B are perspective views of a multi-channel medical device in communication with a machine-readable input device, in accordance with the present invention. FIG. 5A illustrates an infusion pump with a split screen display, having one portion associated with each channel. FIG. 5B illustrates an infusion pump with a screen display for receiving infusion programmable input from a user. The medical device 14 in this example is a multi-channel infusion pump. Those skilled in the art will appreciate that the medical device 14 can be a single channel infusion pump, a multi-channel infusion pump (as shown), a combination thereof, or the like, as desired for a particular application.

Referring to FIG. 5A, the medical device 14 provides a machine-readable input device 130. The machine-readable input device 130 communicates with the medical device 14 to input machine-readable information to the medical device 14. The machine-readable input device 130 can communicate, directly or indirectly, with the medical device 14 via a wireless or hard-wired connection. The machine-readable input device 130 can be a device that is separate from but associated or in communication with the medical device 14. The machine-readable input device 130 can be any sort of data input means, including those adapted to read machine-readable indicia, such as a barcode scanner or handheld personal digital assistant (PDA). Alternatively, the machine-readable input device 130 can be operable to read in other known forms of machine-readable information, such as radio frequency identification tags (RFID), touch memory, digital photography, biometrics, etc.

The medical device 14 is a multi-channel pump having a first channel 132 with first channel machine-readable label 134 and a second channel 136 with a second channel machine-readable label 138. A user of the medical device 14 operates the machine-readable input device 130 to select a channel from one or more channels 132 and 136, by scanning in the associated machine-readable label 134 or 138.

The user selects the desired channel 132 or 136 by using the machine-readable input device 130 to scan a factory or hospital programmed, unique, machine-readable label 134 or 138 that is electronically generated and presented on the screen 122, preferably positioned near the respective channel 132 or 136. Alternatively, the machine-readable labels 134 and 138 are physically affixed to the medical device 14, preferably on or positioned near the channel 132 and 136, respectively. Since the machine-readable labels 134 and 138 are generated and/or can be stored in memory 124 by the medical device 14, the medical device 14 can associate the machine-readable labels 134 and 138 to the channels 132 or 136. The medical device 14 then allows the user to program and activate the selected channel 132 or 136. The user may also manually select the desired channel by touching an appropriate folder tab on the touch screen. The folder tabs are labeled and/or physically arranged on the screen so as to be proximate to the corresponding channel 132 or 136.

In a further aspect of the wireless embodiment, all the medical devices can periodically broadcast a unique wireless device/channel IP address and/or a self-generated unique machine-readable label (for example, a barcode) 134 or 138 that can also be presented on the screen 122. Alternatively, the machine-readable labels 134 and 138 are physically affixed to or posted on the medical device 14. Each medical device will correlate such broadcasted or posted device/channel IP addresses and/or barcodes with a particular patient, who is also identified by a unique machine readable label (not shown) or patient IP address. The user associates the desired pump(s) or channel(s) 132, 136 with the patient by using the machine-readable input device 130 to scan the unique machine-readable labels 134, 138 and the patient's machine readable label. This causes the appropriate pump processor(s) 118 to associate the appropriate pump channel(s) 132, 136 with the patient. Then the pumps or channels can associate, communicate, and coordinate with each other wirelessly.

The graphical user interface program reallocates screen 122 for the medical device 14. Specifically, FIG. 5A illustrates a multi-channel infusion medical device 14 with a split touch screen 122 having a first channel screen portion 140 associated with first channel 132 and a second channel screen portion 142 associated with the second channel 136. Each channel screen portion 140 and 142 presents a subset of the delivery information regarding the respective channels 132 or 136 including without limitation therapeutic agent name, concentration, dose rate, VTBI, and alarm information, in a font size that it is easily readable by a user from a distance such as, for example, from approximately fifteen to twenty feet (4.6-6.2 meters) away. This is what is defined herein as a “far view” delivery screen. The far view delivery screens display subsets of the information found on the relevant “near view” delivery screens. The near view delivery screen displays information such as, drug name, concentration, dose rate, time remaining, VTBI, volume remaining, and alarm name for the highest priority alarm if in an alarm state.

In practice, the delivery screen displays a near view when the user is programming the device as illustrated by FIG. 5B. The near view delivery screen will switch to the far view delivery screen after a predetermined period of time that is predetermined by the manufacturer, configurable by the facility via the drug library, and/or set by the caregiver at the pump, for example after 20 seconds. Often, the user does not want to wait for the predetermined length of time to view the far screen.

Returning to FIG. 5A, the channel screen portion 140 or 142 selected or corresponding to the tab selected expands in area but the size of at least some of the text therein is shrunk. The shrinkage of one of the channel screen portions 140 and 142 and enlargement of its counterpart provides additional space for one or more data display or data entry fields to be placed on screen 122, as shown in FIG. 5B. As discussed below, data displays or data entry fields are placed on screen 122 in space previously occupied by portions of the channel screen portion 140 or 142. This reallocation of space on screen 122 permits the user to enter inputs more easily since the data entry field can be large, preferably at least as large or, more preferably, larger in area than the original channel screen portions 140 and 142 were in the delivery screen mode. Additionally, the reallocation of space on screen 122 provides greater space for presenting information on the channel being adjusted or monitored.

Referring again to FIG. 5A, the medical device 14 includes dedicated or fixed tactile infuser buttons, and images of buttons on the LCD-touch screen 122. The fixed tactile buttons 133, 135, 137, and 139 provide the following functions: LOAD/EJECT button 133 —-opens and closes the cassette carriage; ON/OFF button 135—turns power on and off; ALARM SILENCE button 137—silences a silenceable alarm for a specified period of time, for example two minutes; and EMERGENCY STOP button 139—stops all channels. The LCD color touch screen 122 allows the user to access and use on-screen button images, for example 3D button images, and data entry fields. The touch screen 122 uses a membrane over the LCD display so a single keypress does not cause significant infusion pole movement nor is it mistaken for a double keypress. The touch screen also accommodates a keypress whether the user is wearing wet gloves, dry gloves, or no gloves.

LCD touch screen button images 143, 145, 147, and 149A-149E are located as shown in FIGS. 5A & 5B perform the following functions: Patient Information Tab 143—displays the clinical care area, preselected patient information (including without limitation name, ID number, etc.), and provides access to a more detailed patient information screen; Channel Level Therapy Buttons 145—accessed by button images on the infuser touch screen, are used to select an infusion therapy; Program Level Buttons 147—accessed by pressing areas, drop-down list triangles, boxes or text boxes on the programming screen, are used to select dose parameters of an infusion; and Device Level Buttons 149A-149E at the bottom of the touch screen are used to display and control device level features, including without limitation Mode 149A (for example, Operational or Biomed), Logs 149B, Locks 149C, Settings 149D, and Calculator display 149E. A wireless indicator image 102 displayed at the bottom of the screen 122 indicates that the medical device 14 is connected and ready for communication.

By using the Channel Level Therapy Buttons 145 and the Program Level Buttons 147, the healthcare practitioner can program each individual channel of the pump with specific fluid therapies in a variety of weight- and body surface area-based units such as micrograms/kg/hour, grams/m2/hr, and other delivery specifications for the following modes: Basic Therapy—includes dose calculation, which allows dose rate programming based on volume to be infused (VTBI), drug amount, infusion time and drug concentration and simple rate programming that allows programming of volumetric rate (ml/hr) based upon VTBI and time; Bolus delivery—allows user to program a single uninterrupted discrete delivery based on dose amount and time (the bolus can be delivered from the primary or a secondary container); Piggyback delivery—allows user to program the delivery of a secondary infusion, to be delivered through the same cassette as the primary infusion (the primary infusion is paused until the piggyback VTBI completes); and Advanced Programming. Advanced Programming mode provides various types of programs including: Multistep—which allows a sequential delivery of fluid in up to 10 steps, with fluid volumes and delivery rates programmable for each step based on Rate and Volume or Volume and Time; Variable Time—which allows up to 24 dose calculation steps at specified clock times; intermittent—a calculated dose or step to be delivered at regular intervals; and Taper—a delivery that ramps up and/or ramps down to a plateau rate.

Referring to FIGS. 4 & 5A, the graphical user interface 126 provides channel indicators presented on screen 122. The channel indicators associate on-screen programming, delivery, and alarm information with a particular delivery channel by using graphical depictions such as a channel indication icon 154, 155. The channel indication icon 154 or 155 is a graphical item clearly associating on-screen programming, delivery, and alarm information with a specified associated delivery channel. The channel indication icons 154 and 155 are located on a tab 158 associated with a specified delivery channel of the medical device The channel indication icon 154 or 155 may include but is not limited to a user readable letter or number, a machine-readable indicator 134, or a combination thereof. The graphical user interface program 126 also provides a drip indicator icon 160 and an infusion status icon 156 presented on screen 122.

Referring to FIG. 5B, the screen 122 provides an optional drop-down box 170 for setting an Allow Rate Catch-Up flag to one of an Enabled setting and a Disabled setting at the pump. The drop-down box 170 allows the user to enable or disable the rate catch-up function and to override the default rate catch-up flag provided in the drug library, if desired. When the Allow Rate Catch-Up flag is set to the Enabled setting, the user can enter a catch-up rate factor in the catch-up rate factor value box 172. In this example, the screen 122 also displays a catch-up rate factor limit value 174 and a catch-up rate factor alarm value 176 provided through the drug library. The catch-up rate factor limit value 174 is the maximum catch-up rate factor which the user can enter in the catch-up rate factor value box 172, i.e., the maximum catch-up rate factor or hard limit allowed for the particular therapeutic agent. The catch-up rate factor alarm value 176 is a soft limit on the catch-up rate factor. In one example, the screen 122 will provide an alarm when the user enters a value in the catch-up rate factor value box 172 which exceeds the catch-up rate factor alarm value 176, but the infusion pump will accept the catch-up rate factor after the user acknowledges the alarm or indicates a decision to override the soft limit as long as the catch-up rate factor does not exceed the hard limit or catch-up rate factor limit value 174. Those skilled in the art will appreciate that the catch-up rate factor limit value 174 and the catch-up rate factor alarm value 176 can be omitted or set to a high value as desired for a particular application. In one embodiment, the default values for the Allow Rate Catch-Up flag setting, catch-up rate factor value box 172, catch-up rate factor limit value 174, and/or catch-up rate factor alarm value 176 can be loaded into the infusion pump from a remote computer as part of a drug library editing program such as HOSPIRA MEDNET™ software. In another embodiment, the default values for the Allow Rate Catch-Up flag, catch-up rate factor value box 172, catch-up rate factor limit value 174, and/or catch-up rate factor alarm value 176 can be loaded into the infusion pump by the user at the infusion pump. In another hybrid embodiment, the default values can be established in a drug library downloaded to the pump and, if allowed by a setting in the drug library, later overridden or modified by the user at the pump. In another embodiment, the entire catch-up rate behavior can be predetermined by the manufacturer and hard coded into the pump, without any user customization being allowed.

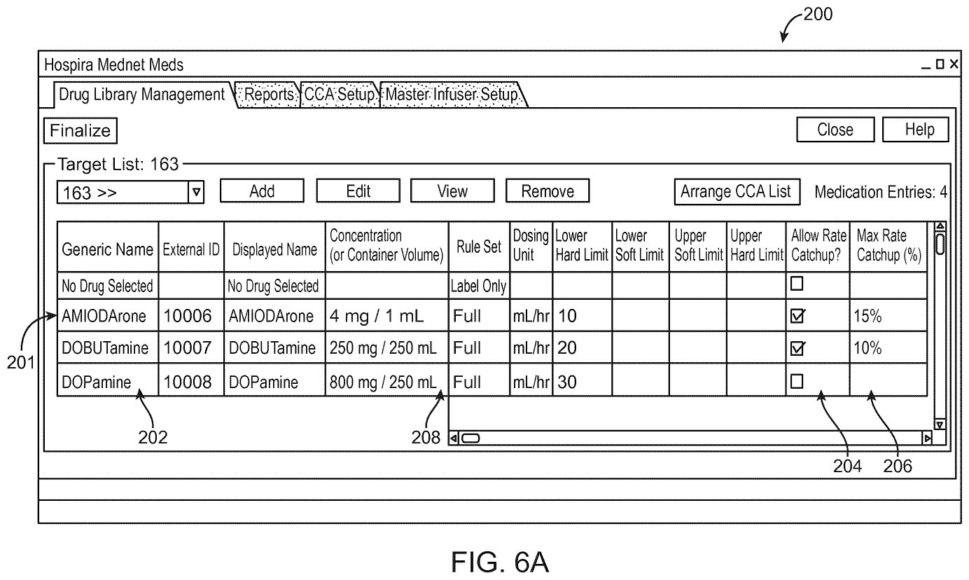

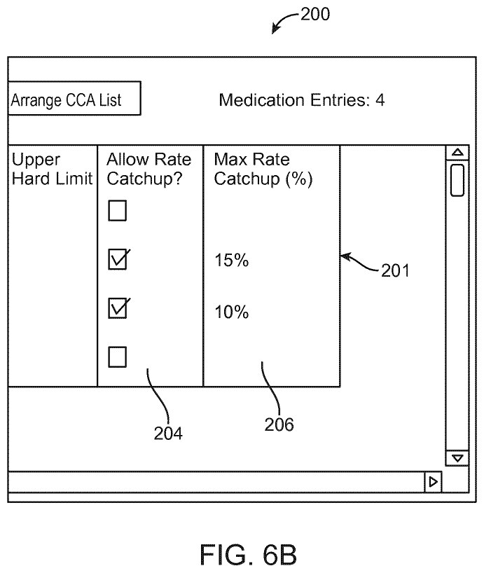

FIGS. 6A & 6B are screen shots of a graphical user interface and detail of the graphical user interface, respectively, for configuring a drug library, in accordance with the present invention. The graphical user interface 200 can be displayed on the display/input device 38 of the MMU 12 (as shown in FIG. 2) and used to receive data for creating or updating the drug library, such as the catch-up rate factor, Allow Rate Catch-up flag setting, maximum catch-up rate factor setting, maximum catch-up rate factor alarm setting, and the like.

Referring to FIGS. 6A & 6B, the graphical user interface 200 includes a table 201 to receive different drugs and therapeutic agents in the drug library database. Drug list 202 includes a list of the names of the drugs in the drug library, which could be the generic names, brand names or both. The drug list can include multiple entries for the same drug but different concentrations or clinical uses (cardiac, renal, pediatric). The Allow Rate Catch-Up Flag list 204 includes the allow rate catch-up flag setting for each drug/concentration/use entry (“drug entry” for short) in the drug list 202 to determine whether rate catch-up is allowed for the particular drug entry. In addition, the Allow Rate Catch-up flag setting can be a function of pump type and/or clinical care area location. The maximum rate catch-up list 206 includes the maximum catch-up rate factor setting for each drug entry in the drug list 202 for which rate catch-up is allowed. The maximum catch-up rate factor setting also can be a function of pump type and clinical care area location. In one embodiment, allowable maximum catch-up rate factors are regular linear percentages at predetermined intervals, e.g., 5%, 10%, 15%, 20%, et cetera. The table 201 can also include other parameters that constrain or limit the drug maximum catch-up rate such as the normal global constraints on rate already configured via the MMU 12 and HOSPIRA MEDNET™ software (Lower Hard Limit, Lower Soft Limit, Upper Soft Limit, and/or Upper Hard Limit). The maximum catch-up rate factor alarm setting and other drug maximum catch-up rate limits for each drug entry also can be a function of pump type and clinical care area location.

The catch-up rate factor is a simple percentage applied to the desired infusion rate to obtain a catch-up infusion rate when the actual accumulated infusion volume is less than the expected accumulated infusion volume. In some cases the desired infusion rate is input directly as a rate or volume per unit of time, such as ml/hr. In other cases the desired infusion rate is a calculated value based upon a dose and the weight or body surface area of the patient. For example, a dose of 10 ml/kg/hr can be prescribed for a patient who weighs 100 kg. Thus, the desired infusion rate would be calculated as 1000 ml/hr. In other cases the desired infusion rate is calculated based upon the dose and concentration of drug in the container. For example, if a dose of 10 mcg/hr is prescribed to be delivered from a 1000 ml container of fluid that has a concentration of 100 mcg of the drug, then the desired infusion rate is calculated at 100 ml/hr. There are other alternative dosing units that are known in the art to provide calculated desired infusion rates. In one embodiment, the catch-up rate factor is added to the desired infusion rate. In another embodiment, the catch-up rate factor (for example 1.05) is multiplied by the desired infusion rate. In one embodiment, allowable catch-up rate factors are regular linear percentages at predetermined intervals, e.g., 5%, 10%, 15%, 20%, et cetera to make it easier for the user to select a catch-up rate factor. The catch-up rate factor applies a linear adjustment to the desired infusion rate and does not rely on any input from physiological factors of the patient. Thus, the configurable closed loop delivery rate catch-up is straightforward and does not rely on complex algorithms or control schemes. Instead the feedback mechanism of this algorithm is based only on the measured versus expected accumulated volume delivered over time by the pump. The new rate Y is determined by a simple single order equation X+AX or AX; where A equals the catch-up rate factor as described above.

The Allow Rate Catch-up flag setting as a function of pump type can account for different pump types, makes and models, as well as uses for which various pump types are employed. In one example, one pump type using a cartridge and driving a plunger with a stepper motor can be used for general infusion such as saline solutions or the like, so that there is little risk in allowing rate catch-up. In another example, another pump type using a prefilled syringe can be used for analgesics or opiates, so that it may not be desirable to allow rate catch-up. Other pump types may have multiple uses or therapies and it may be desirable to control the enablement of the catch-up rate feature for each of the plurality of uses available with such a pump type.

The Allow Rate Catch-up flag setting can be a function of clinical care area location. In one example, an infusion pump used in a treatment area in which the patients are in serious or critical condition, such as an emergency or operating room, may not want to allow rate catch-up. In another example, an infusion pump used in a treatment area in which the patients are in good condition may want to allow rate catch-up.

Those skilled in the art will appreciate that the table 201 can also include other data as desired for a particular application. The table 201 can include other exemplary columns 208 for additional data for the different drugs, such as External Drug ID Numbers, Drug Display Names, Drug Concentration/Container Volume, Selected Drug Rule Set (Label Only, Limited, Full), Drug Dosing Unit, Drug Dosing Limits (Lower Hard Limit, Lower Soft/Alarm Limit, Upper Soft/Alarm Limit, and/or Upper Hard Limit) or the like.

The drug library provides flexibility for various combinations of parameters as desired for a particular application. The drug library can have different Allow Rate Catch-up flag settings for different drugs or drug entries in the drug library. The drug library can have different maximum permissible catch-up rate factor settings for different drugs in the drug library. The drug library can have a given drug listed in multiple different clinical care areas (CCAs) in the drug library with at least one of different Allow Rate Catch-up flag settings and different maximum catch-up rate factor settings for a the given drug.

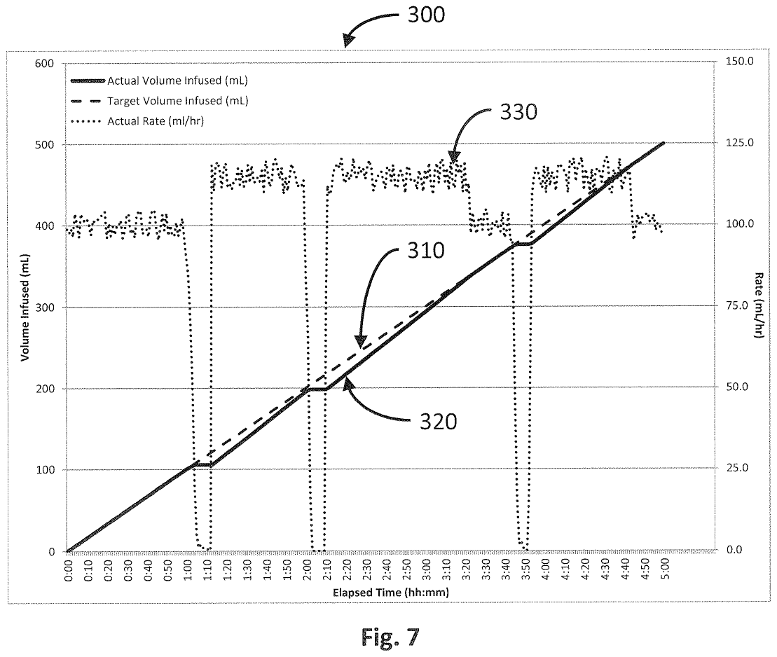

FIG. 7 is a graph of an infusion volume and infusion rate versus time modeled for an infusion with an infusion pump employing configurable closed loop delivery rate catch-up, in accordance with the present invention. The graph 300 includes the expected accumulated infusion volume 310, the actual accumulated infusion volume 320, and the infusion rate 330.

The expected accumulated infusion volume 310 increases linearly at a desired infusion rate of 100 ml per hour. The actual accumulated infusion volume 320 increases linearly from time 0:00 until time 1:00 at the originally programmed or desired infusion rate 330 of 100 ml per hour. At time 1:00, the infusion is interrupted so that the infusion rate 330 remains approximately zero and the actual accumulated infusion volume 320 remains about 100 ml until time 1:15, when the infusion resumes. At time 1:15, the actual accumulated infusion volume 320 is less than the expected accumulated infusion volume 310, so the infusion rate is increased by the catch-up rate factor of 15% and the infusion resumes at a catch-up infusion rate of 115 ml per hour from time 1:15 to time 2:00. At time 2:00, the actual accumulated infusion volume 320 has not quite yet caught up with the expected accumulated infusion volume 310 and the infusion is once again interrupted. The infusion rate 330 remains approximately zero and the actual accumulated infusion volume 320 remains about 200 ml until time 2:10, when the infusion resumes. From time 2:10 until time 3:20, the infusion is delivered at the catch-up infusion rate of 115 ml per hour until the actual accumulated infusion volume 320 equals the expected accumulated infusion volume 310 at time 3:20, when the infusion rate 330 is reduced to the originally programmed or desired infusion rate of 100 ml per hour. At time 3:45, the infusion is once again interrupted so that the infusion rate 330 remains approximately 0 and the actual accumulated infusion volume 320 remains at about 375 ml until the infusion resumes at time 3:55. From time 3:55 until time 4:40, the infusion is delivered at the catch-up infusion rate of 115 ml per hour until the actual accumulated infusion volume 320 equals the expected accumulated infusion volume 310 at time 4:40, when the infusion rate 330 is reduced to the originally programmed or desired infusion rate of 100 ml per hour. Thus, the desired accumulated volume of 500 ml has been delivered by the scheduled time 5:00 in spite of three interruptions in the infusion.

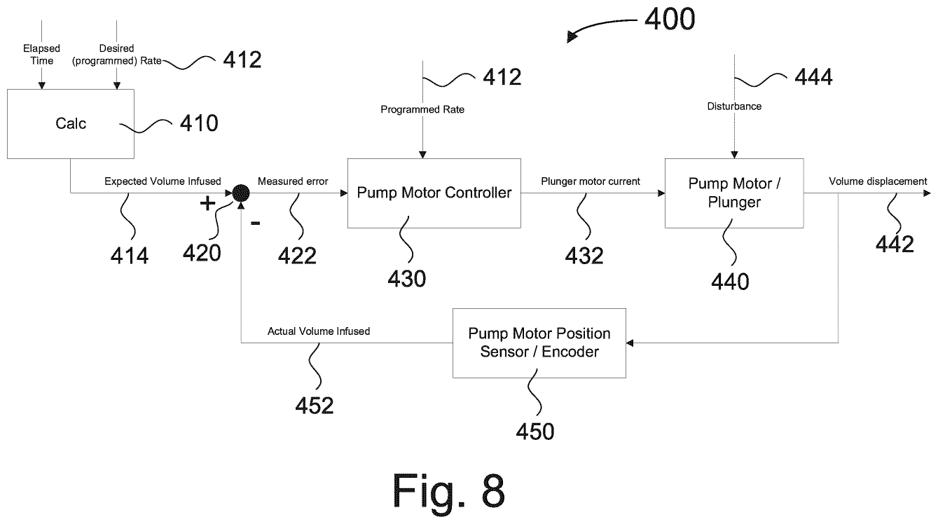

FIG. 8 is a block diagram of a control model for an infusion pump employing configurable closed loop delivery rate catch-up, in accordance with the present invention. The control model 400 includes an infusion volume calculator 410, a volume comparator 420, a pump controller 430, a pump drive 440, and a flow integrator 450. The infusion volume calculator 410 receives a desired infusion rate signal 412 and generates an expected accumulated infusion volume signal 414 from the originally programmed desired infusion rate signal 412 and the elapsed time. The volume comparator 420 receives the expected infusion volume signal 414 and an actual accumulated infusion volume signal 452, and generates a volume error signal 422 from the expected accumulated infusion volume signal 414 and the actual accumulated infusion volume signal 452. The pump controller 430 also receives the desired infusion rate signal 412 and the accumulated volume error signal 422, and generates a pump drive signal 432 from the desired infusion rate signal 412 and the accumulated volume error signal 422. The pump drive 440 receives the pump drive signal 432 to deliver the infusion 442. For modeling purposes, the pump drive 440 is subject to disturbances 444 which can cause or result in interrupted delivery of the infusion 442. The disturbance may include but are not limited to stoppages due to alarms, occlusions and other faults. The flow integrator 450 is operable to monitor the pump drive 440 and/or the infusion 442 and generate the actual accumulated infusion volume signal 452. In one embodiment, the pump drive 440 moves the plunger in a syringe and the flow integrator 450 senses pump drive/plunger position. In another embodiment, the pump drive 440 is a stepper motor and the flow integrator 450 counts pump strokes or motor steps. In yet another embodiment, the pump drive 440 is a rotary pump and the flow integrator 450 counts pump rotations. The present invention could also be applied with a drip counting device to provide the necessary feedback concerning the actual flow rate and/or accumulated volume. Returning to discussion of the embodiment for driven pumps, the pump drive signal 432 is a function of the desired infusion rate signal 412 multiplied by a catch-up rate factor when the volume error signal 422 meets (equals and/or exceeds) a threshold that indicates that actual accumulated infusion volume is less than expected accumulated infusion volume, to catch up interrupted delivery of an infusion. The pump drive signal 432 is a function of the desired infusion rate signal 412 alone or returns to the originally programmed or set rate when the accumulated volume error signal 422 indicates that the actual accumulated infusion volume is greater than or equal to the expected accumulated infusion volume.

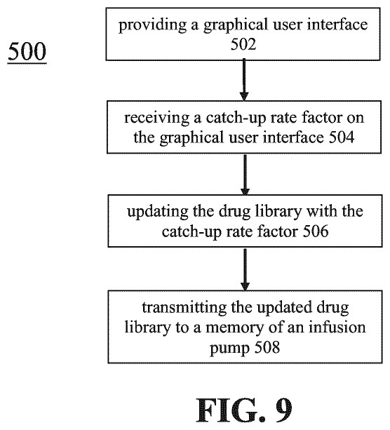

FIG. 9 is a flowchart of a method for configuring a drug library for use with an infusion system employing configurable closed loop delivery rate catch-up, in accordance with the present invention. The method 500 includes providing a graphical user interface 502 for modifying a drug library of the medication management unit; receiving a catch-up rate factor on the graphical user interface 504; updating the drug library with the catch-up rate factor 506; and transmitting the updated drug library to a memory of an infusion pump 508. The method 500 can be performed on a medication management unit having a processing unit and a storage medium coupled to the processing unit, the storage medium containing programming code executable by the processing unit to perform the steps of the method 500.

Those skilled in the art will appreciate that the drug library can include additional settings for a method for configuring a drug library for use with an infusion system employing configurable closed loop delivery rate catch-up as desired for a particular application. In one embodiment, the drug library can further include an Allow Rate Catch-up flag setting having one of an Enabled setting and a Disabled setting, the Allow Rate Catch-up flag setting being a function of a parameter selected from the group consisting of drug/concentration/use entry, pump type, and clinical care area location. In another embodiment, the drug library can further include a maximum catch-up rate factor setting having a numerical value, the maximum catch-up rate factor setting being a function of a parameter selected from the group consisting of drug entry, pump type and clinical care area location. In yet another embodiment, the drug library can further include a maximum catch-up rate factor alarm setting having a numerical value, the maximum catch-up rate factor alarm setting being a function of a parameter selected from the group consisting of drug entry, pump type and clinical care area location.

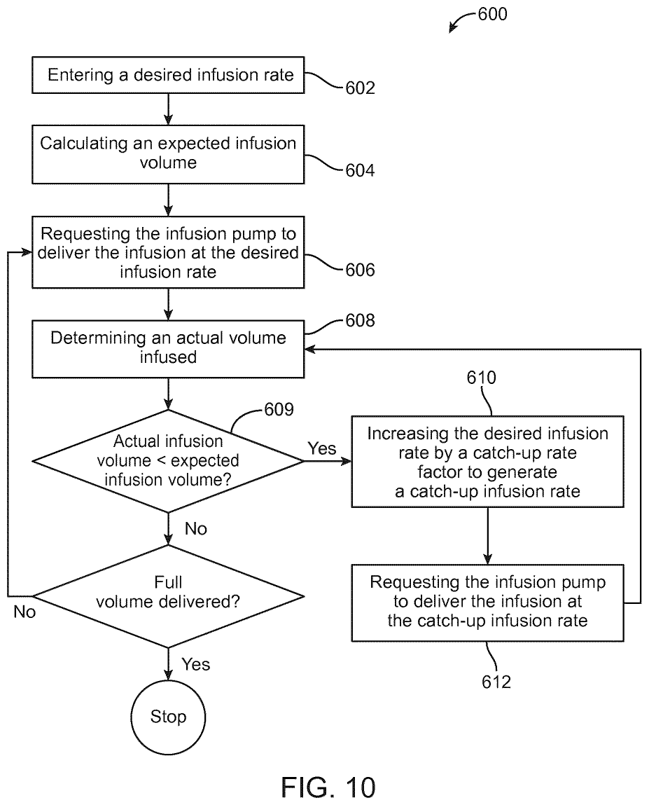

FIG. 10 is a flowchart of a method for operating an infusion pump employing configurable closed loop delivery rate catch-up, in accordance with the present invention. The method 600 includes entering a desired infusion rate 602 for the infusion pump; calculating an expected accumulated infusion volume 604 as a function of time from the infusion rate; requesting the infusion pump to deliver the infusion at the desired infusion rate 606; determining an actual accumulated infusion volume 608 at a particular time; and determining whether the actual accumulated infusion volume is less than the expected accumulated infusion volume 609. When the actual accumulated infusion volume is less than the expected accumulated infusion volume, the method 600 continues with increasing the desired infusion rate by a catch-up rate factor to generate a catch-up infusion rate 610 and requesting the infusion pump to deliver the infusion at the catch-up infusion rate 612. When the actual accumulated infusion volume is not less than the expected accumulated infusion volume, the method 600 continues monitoring the output of the pump and with delivering the infusion at the originally programmed or desired infusion rate. The method 600 can also request the infusion pump to deliver the infusion at the originally programmed or desired infusion rate after delivery of the infusion at the catch-up infusion rate 612 when the actual accumulated infusion volume is equal to the expected accumulated infusion volume. Once the actual accumulated infusion volume is equal to or greater than the expected accumulated infusion volume, the method 600 continues by determining whether the full accumulated volume prescribed or programmed has been delivered or not. The method 600 stops if the full volume has been delivered, or loops back to continue with step 606 if more volume remains to be delivered.

The method 600 can also allow the user at the infusion pump to provide input. The method 600 can include a user at the infusion pump inputting an Allow Rate Catch-up flag setting to a Disabled setting to disable the increasing and the delivery rate catch-up function. The method 600 can include the user at the infusion pump inputting the catch-up rate factor to a desired value. In one embodiment, the catch-up rate factors are regular linear percentages at predetermined intervals, e.g., 5%, 10%, 15%, 20%, et cetera. In other embodiments, the drug library editor or the pump may allow the user more flexibility to customize the values and the intervals between them.

The method 600 may or may not require user action before applying the catch-up infusion rate if desired for a particular application. In one embodiment, the requesting the infusion pump to deliver the infusion at the catch-up infusion rate 612 can occur automatically, without user action, by increasing the desired infusion rate by a catch-up rate factor to generate a catch-up infusion rate 610. In another embodiment, the method 600 can include annunciating an alarm or warning before increasing the desired infusion rate by a catch-up rate factor to generate a catch-up infusion rate 610, and requiring the user acknowledge the alarm and confirm that the catch-up rate behavior is desired before the requesting the infusion pump to deliver the infusion at the catch-up infusion rate 612. In another embodiment, the method 600 can include annunciating an alarm or warning after increasing the desired infusion rate by a catch-up rate factor to generate a catch-up infusion rate 610, and allowing the user to confirm or reject the catch-up rate behavior. If the catch-up rate behavior is rejected, the infusion will revert to the originally programmed infusion rate.

The method 600 can also provide limits and alarms in response to user input. In one embodiment, the method 600 further includes rejecting the input catch-up rate factor when the desired value is greater than a maximum catch-up rate factor setting. In another embodiment, the method 600 further includes providing an alarm when the desired value is greater than a maximum catch-up rate factor alarm setting. In yet another embodiment, the increasing the desired infusion rate by a catch-up rate factor in the method 600 includes receiving an alarm when at the particular time the actual accumulated infusion volume is less than the expected accumulated infusion volume; and acknowledging the alarm prior to increasing the desired infusion rate by a catch-up rate factor to generate a catch-up infusion rate.

The method 600 can be performed on an infusion pump having a processor and the memory coupled to the processor, the memory containing programming code executable by the processor to perform the steps of the method 600. In one embodiment, the infusion pump 14 can be in electronic communication with a medication management unit 12 and the catch-up rate factor can be part of an updated drug library transmitted from the medication management unit 12 and received at the medical device 14.

Terms of equality and inequality (less than, greater than) as used herein as commonly used in the art, i.e., accounting for uncertainties present in measurement and control systems. Thus, such terms can be read as approximately equal, approximate less than, and/or approximately greater than. In one example, two values can be considered equal when they are within 5% of each other. In other aspects of the invention, an acceptable threshold of deviation or hysteresis can be established by the pump manufacturer, the editor of the drug library, or the user at the pump.

While the embodiments of the invention disclosed herein are presently considered to be preferred, various changes and modifications can be made without departing from the scope of the invention. The scope of the invention is indicated in the appended claims, and all changes that come within the meaning and range of equivalents are intended to be embraced therein.

1. -16. (canceled)

17. An infusion system comprising one or more hardware processors configured to:
receive a desired infusion rate;
calculate an expected accumulated infusion volume as a function of time from the desired infusion rate;
activate delivery of an infusion at the desired infusion rate;
determine an actual accumulated infusion volume at a particular time;
generate a catch-up infusion rate using the desired infusion rate and a catch-up rate based on a comparison of the actual accumulated infusion volume and the expected accumulated infusion volume; and
change delivery of the infusion at the catch-up infusion rate.

18. The infusion system of claim 17, wherein the one or more hardware processors are further configured to request an infusion pump to deliver the infusion at the desired infusion rate after delivery of the infusion at the catch-up infusion rate when the actual accumulated infusion volume is equal to the expected accumulated infusion volume.

19. The infusion system of claim 17, wherein the desired infusion rate is receiving from a drug library.

20. The infusion system of claim 17, wherein the one or more hardware processors are further configured to disable delivery at the catch-up infusion rate based on information received from a drug library.

21. The infusion system of claim 17, wherein the one or more hardware processors are further configured to disable delivery at the catch-up infusion rate

22. The infusion system of claim 17, wherein the one or more hardware processors are further configured to receive input of an Allow Rate Catch-up flag setting from a user at the infusion.

23. The infusion system of claim 17, wherein the one or more hardware processors are further configured to receive input of a maximum permissible catch-up rate.

24. The infusion system of claim 17, wherein the generation of the catch-up infusion rate does not rely on physiological factors of a patient.

25. The infusion system of claim 17, wherein the delivery of the infusion is changed to the catch-up infusion rate based on a location of an infusion pump.

26. The infusion system of claim 17, wherein the one or more hardware processors are configured to generate an alarm and request user input before changing the delivery of the infusion at the catch-up infusion rate.

27. An infusion method comprising:
receiving a desired infusion rate;
calculating an expected accumulated infusion volume as a function of time from the desired infusion rate;
activating delivery of an infusion at the desired infusion rate;
determining an actual accumulated infusion volume at a particular time;
generating a catch-up infusion rate using the desired infusion rate and a catch-up rate based on a comparison of the actual accumulated infusion volume and the expected accumulated infusion volume; and
changing delivery of the infusion at the catch-up infusion rate.

28. The infusion method of claim 27, further comprising delivering the infusion at the desired infusion rate after delivery of the infusion at the catch-up infusion rate when the actual accumulated infusion volume is equal to the expected accumulated infusion volume.

29. The infusion method of claim 27, wherein the desired infusion rate is receiving from a drug library.

30. The infusion method of claim 27, further comprising disabling delivery at the catch-up infusion rate based on information received from a drug library.

31. The infusion method of claim 27, further comprising disabling delivery at the catch-up infusion rate based on a location in a hospital.

32. The infusion method of claim 27, further comprising receiving an input of an Allow Rate Catch-up flag setting from a user at the infusion.

33. The infusion method of claim 27, further comprising receiving an input of a maximum permissible catch-up rate.

34. The infusion method of claim 27, wherein the generation of the catch-up infusion rate does not rely on physiological factors of a patient.

35. The infusion method of claim 27, wherein the delivery of the infusion is changed to the catch-up infusion rate based on a location of an infusion pump.

36. The infusion method of claim 27, further comprising generating an alarm and request user input before changing the delivery of the infusion at the catch-up infusion rate.
